(19) United States
(12) Patent Application Publication
(10) Pub. No.: US 2005/0074418 A1
(43) Pub. Date: Apr. 7, 2005

(54) Process for permanently reshaping the hair, comprising a step of in situ precipitation of an anionic or amphoteric polymer

(76) Inventors: Catherine Campain, Paris (FR); Priscille Devin-Baudoin, Vanves (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/913,399

(22) Filed: Aug. 9, 2004

Related U.S. Application Data
(60) Provisional application No. 60/505,158, filed on Sep. 24, 2003.

(30) Foreign Application Priority Data
Aug. 7, 2003 (FR) 03 09737
May 14, 2004 (FR) 04 05271

(51) Int. Cl. A61K 7/09

(52) U.S. Cl. 424/70.2

(57) ABSTRACT

The present disclosure relates to a process for permanently reshaping hair, comprising: applying, to an area to be reshaped, a solution comprising at least one polymer chosen from anionic and amphoteric polymers in a medium comprising water and, optionally, at least one cosmetically acceptable solvent, applying, to the area treated in step (a), a reducing composition comprising at least one reducing agent that is capable of cleaving disulfide linkages of the hair and a sufficient amount of ions chosen from metal cations and ammonium ions to precipitate in situ the at least one polymer, shaping the hair in the area treated, followed by a leave-in time for the reducing composition, applying, to the area treated in steps (a) and (b), a fixing composition comprising at least one oxidizing agent, followed by a leave-in time for the fixing composition, and rinsing the hair, and also to a multi-component agent and a multi-compartment kit for the same.

This application claims benefit of U.S. Provisional Application No. 60/505,158, filed Sep. 24, 2003.

The present disclosure relates to a process for permanently reshaping the hair, comprising a step of in situ precipitation of a water-soluble polymer, as well as a multi-component agent and a multi-compartment kit for performing such a process.

One of the techniques currently used for permanently reshaping keratin material, e.g., human hair, comprises treating it successively with a reducing agent capable of cleaving the disulfide bridges of keratin, and then with an oxidizing agent that re-establishes the disulfide bridges while at the same time holding the hair under a mechanical constraint, for example, using curlers.

The traction exerted by the curlers makes it possible to obtain detachment of the roots, but also involves waving the hair. Now, many women wish to increase the volume of their head of hair without, however, having curly hair.

It is, admittedly, possible to use large-sized curlers to attenuate the curly appearance and simply obtain gentle waving, but the hold of such permanent reshaping is often not very good.

Another drawback of known permanent-reshaping techniques lies in the possible sensitization of the hair resulting from the repeated treatment of the hair with strong oxidizing and reducing agents.

To overcome at least one of these drawbacks, it is known to use gelled reducing compositions, which allow the product to be localized on the roots and thus prevents chemical degradation of the hair over its entire length. The use of a gelled reducing composition, however, does not dispense with the step of rolling up the hair on curlers, and this technique consequently remains possibly long and difficult to perform by a user and also may give a reshaping result that is not very long-lasting.

A certain number of attempts have been made to permanently reshape the hair without using mechanical shaping devices, such as curlers. For instance, Patent Application No. WO 99/18922 discloses a process for permanently reshaping the hair using a thickened reducing composition that has moderate adhesive power. This reducing composition allows the hair to be shaped without applying any tension constraint. The oxidizing composition is applied directly to the hair treated with the reducing composition, without intermediate rinsing intended to remove the reducing agent. WO 99/18922, however, does not appear to disclose the partial reshaping of the hair, i.e., limited to certain areas, for example to the area close to the roots.

Further, Patent Application No. WO 01/95868 discloses a process for permanently reshaping the hair that is limited to certain areas, for example, to the area close to the roots, which does not use mechanical means for placing the hair under tension, such as curlers. The hold of the shape given to the hair during the reduction and oxidation phases is ensured by the reducing composition itself, which contains a wax, absorbent hydrophilic particles, such as particles of flour, and a reducing agent absorbed by the hydrophilic particles. This process is relatively complex, since it involves melting the wax in a suitable heating device and mixing the molten wax with the absorbent hydrophilic particles and the reducing agent. The application of the hot mixture to the hair requires a certain amount of dexterity on the part of the hair stylist and can be difficult for a user to perform alone. Moreover, relatively substantial stripping of the hair color has been observed when this technique is used for permanently reshaping dyed hair.

The process disclosed herein is much simpler to perform than the above-described process and does not require the prior preparation of the reducing composition with a heating device. In the process of the present disclosure, the hold of the shape given to the hair during the reduction and oxidation steps is not achieved by the solidification of a fluid or malleable substance, but by in situ precipitation of a polymer applied to the hair in solution form.

The present disclosure involves the successive application of three compositions (a polymer composition, a reducing composition and a fixing composition) and manual shaping of the hair, not requiring any mechanical tensioning devices.

One aspect of the present disclosure is a process for permanently reshaping hair, comprising
(a) applying, to an area to be reshaped, a solution of at least one polymer chosen from anionic and amphoteric polymers in a medium comprising water and, optionally, at least one cosmetically acceptable solvent,
(b) applying, to the area treated in step (a), at least one reducing composition comprising a reducing agent that is capable of cleaving disulfide linkages of the hair, and a sufficient amount of ions chosen from metal cations and ammonium ions to precipitate in situ the at least one polymer,
(c) shaping the hair in the area treated, followed by a leave-in time for the reducing composition,
(d) applying, to the area treated in steps (a) and (b), a fixing composition comprising at least one oxidizing agent, followed by a leave-in time for the fixing composition, and
(e) rinsing the hair.
   In one embodiment, the steps of the process are carried out in sequential order as outlined above.

Another aspect of the disclosure is a multi-component agent for permanently reshaping the hair, comprising
a first component comprising a solution comprising at least one polymer chosen from anionic and amphoteric polymers in a medium comprising water and, optionally, at least one cosmetically acceptable solvent that is capable of forming a solid precipitate in the presence of a sufficient amount of ions chosen from metal cations and ammonium ions,
a second component comprising at least one reducing composition comprising at least one reducing agent that is capable of cleaving disulfide linkages of the hair, and a sufficient amount of of ions chosen from metal cations and ammonium ions to precipitate the at least one polymer of the first component, and
a third component comprising an oxidizing composition comprising at least one oxidizing agent capable of re-establishing the disulfide linkages of the hair.

This multi-component agent may be, for example, in the form of a multi-compartment kit comprising at least three compartments comprising, respectively, the first, second and third components above, packaged and sold in the same packaging.

The cosmetically acceptable organic solvents that may be used herein to dissolve the anionic and/or amphoteric polymers may be liquid at room temperature and at atmospheric pressure. These solvents are, for example, monoalcohols, polyols or ethers of these alcohols or polyols. Examples that may be mentioned include ethanol, isopropanol, glycerol, propylene glycol, and propylene glycol monomethyl ether. In one embodiment, ethanol may be used.

The first component comprising a solution of at least one polymer chosen from anionic and amphoteric polymers may be in a thickened, or even gelled form, allowing an application targeted to certain areas of the hair.

The anionic and/or amphoteric polymers used in the first component of the present disclosure may be soluble in the liquid medium used. Without wishing to be bound by any theory, the inventors believe that after applying the at least one polymer solution to the hair, the ionic strength of the medium is greatly increased by adding the reducing composition highly charged with metal cations and/or ammonium ions (in the form of salts). This then produces a contraction of the macromolecular ball, finally resulting in precipitation of the at least one polymer.

The ionic strength required to obtain the in situ precipitation of the at least one polymer depends on a certain number of factors, such as the number of charges of the at least one polymer, the more or less hydrophobic nature of the at least one polymer in its uncharged form, the molecular mass of the at least one polymer, the amount of the at least one polymer in the first component, and/or the pH of the medium obtained by the in situ mixing of the first and second components.

This ionic strength may be provided by mineral or organic salts present in the reducing composition of the second component, but also by the at least one reducing agent itself when it is in salt form.

The metal cations and/or ammonium ions in the applied reducing composition (of the second component) may be present in an amount greater than 0.3 M; the inventors have found that this amount is sufficient to obtain a deposit of precipitated polymer of a satisfactory quality, allowing the shape of the head of hair to be held during the reduction and oxidation steps. The amount of the metal cations and/or ammonium ions may also range from, for example, 0.5 to 6 M.

It is possible that any natural or synthetic, anionic or amphoteric polymer may be used.

One category of the at least one polymer that may be used for the process of the disclosure is formed by the synthetic copolymers obtained by polyaddition of a mixture of anionic monomers and of nonionic monomers, or by polyaddition of a mixture of anionic, nonionic and cationic monomers.

The anionic monomers used for the synthesis of such copolymers are, for example, chosen from acrylic acids, methacrylic acids, 2-acrylamidomethylpropanesulfonic acids, crotonic acids, itaconic acids, and mixtures thereof.

Examples of nonionic monomers that may be mentioned include C₁₋₄ alkyl acrylates, C₁₋₄ alkyl methacrylates, acrylamides, vinyl acetates, vinyl para-tert-butylbenzoates, methacrylamides, N-mono(C₁₋₈ alkyl)acrylamides, N-vinylformamides, N-vinylacetonamides, hydroxypropyl acrylates, and hydroxypropyl methacrylates.

The cationic monomers that may be used in combination with the anionic and nonionic monomers for the synthesis of amphoteric copolymers that may be used in the present disclosure are chosen, for example, from di(C₁₋₄ alkyl)diallylammonium salts and the compounds of formula (I) 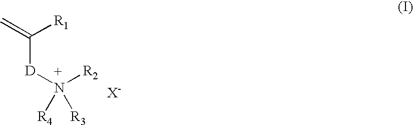
wherein

R₁ is chosen from a hydrogen atom and a methyl group,

R₂ and R₃, which may be identical or different, are chosen from hydrogen atoms and linear and branched C₁₋₄ alkyl groups,

R₄ is chosen from a hydrogen atom, a linear or branched C₁₋₄ alkyl group, and an aryl group,

D comprises the following unit:
YₙA
wherein Y is chosen from amide, ester, urethane and urea functions,

A is chosen from a linear, branched and cyclic C₁₋₁₀ alkylene groups, optionally substituted or interrupted with a ring chosen from divalent aromatic and heteroaromatic rings, optionally interrupted with a hetero atom chosen from O, N, S and P, and optionally comprising at least one function chosen from ketone, amide, ester, urethane and urea functions,

n is 0 or 1, and

X⁻ is an anionic counterion, such as a halide or sulfate ion.

For example, copolymers that may be mentioned include, but are not limited to,
copolymers of (meth)acrylic acid and of a C₁₋₆ alkyl (meth)acrylate (INCI name: acrylates copolymer), sold, for example, under the name Amerhold® DR-25 by the company Amerchol,
copolymers of vinyl acetate, of vinyl para-tert-butylbenzoate and of crotonic acid (INCI name: VA/Vinyl Butylbenzoate/Crotonates Copolymer) sold, for example, under the name Mexomer® PW by the company Chimex, or
copolymers of octylacrylamide, of tert-butylaminoethyl methacrylate and of at least one monomer chosen from acrylic and methacrylic acids and C₁₋₆ alkyl esters thereof (INCI name: octylacrylamide/acrylates/butylaminoethyl/methacrylate copolymer) sold, for example, under the name Amphomer® by the company National Starch.

The anionic and/or amphoteric polymers that may be used herein, however, are in no way limited to the copolymers obtained by polyaddition as described above. It may also be envisaged to use polycondensates such as polyesters, polyamides or polyurethanes, or copolymers and derivatives thereof such as silicone copolymers, bearing a sufficient number of anionic charges and possibly of cationic charges to be soluble in the liquid medium of the first component and hydrophobic enough to form a solid precipitate in the case of an increase in the ionic strength.

For example, the anionic and/or amphoteric polymers that may be mentioned include, but are not limited to:
the product Luviset® Si-P.U.R.A (proposed INCI name: Polyurethane-6), sold by the company BASF Aktiengesellschaft, which is a neutralized silicone polyurethane, in aqueous-alcoholic solution, resulting from the polycondensation of isophthalic acid, of adipic acid, of hexylene glycol, of neopentyl glycol, of dimethylolpropionic acid, of isophorone diisocyanate and of bis(ethylaminoisobutyl) dimethicone, or
the product sold by the company Amerchol under the name Viscophobe® DB-1000 (INCI name: Polyacrylate-3).

The anionic or amphoteric polymer, for example, is present in the solution of the first component in an amount that is sufficient to allow the formation of a deposit of precipitated polymer capable of rigidifying the keratin fibers, i.e., the hair, for the time required to destroy and reconstruct the disulfide linkages of the hair. The anionic and/or amphoteric polymer is present in an amount at least equal to 5% and for example, ranging from 7% to 30%, by weight relative to the total weight of the solution applied in step (a).

To increase the ionic strength of the reduction medium down to a sufficient value, any organic or mineral salt that does not interfere with the reduction reaction of the disulfide bridges may, in principle, be used. For example, ammonium salts and alkali metal salts may be used.

When the cations are provided by the reducing agent (in salt form), this salt comprises at least one carboxylate function. For example, the reducing agent (in salt form) may be chosen from ammonium thioglycolate and ammonium thiolactate.

The metallic or ammonium cations may also be derived from mineral hydroxides. Mineral salts that may be used, include, but are not limited to, alkali metal halides, such as sodium chloride. The organic salts may be, for example, ammonium citrate, and the mineral hydroxides may be sodium hydroxide (NaOH) or aqueous ammonia (NH₄OH).

The reduction step of the process of the present disclosure is, for example, performed at a pH ranging from 7 to 11 and further, for example, from 7.5 to 9.5.

A person skilled in the art will know how to select the pH modifiers and buffer agents that are suitable for adjusting the pH of the first and second components to an adapted value.

The at least one reducing agent of the reducing composition may be any known reducing agent, chosen from those usually used in the field of the permanent reshaping of the hair, such as sulfites, bisulfites, and thiols. Among these reducing agents, cysteine, cysteamine, thiolactic acid, thioglycolic acid, and the cosmetically acceptable salts thereof, such as ammonium thioglycolate, may be used.

The at least one reducing agent is present in an amount ranging from 1% to 25% and for example, from 1% to 10%, by weight relative to the total weight of the reducing composition.

After application of the reducing composition charged with metallic and/or ammonium cations (in the form of mineral or organic salts) and precipitation of the polymer, the hair may be given the desired shape by simply using the hands or by a mechanical device for easily holding longer locks of hair in place, such as clips, combs, hair slides, tie-rolls and elastic bands. This setting in place may be performed by the user and may not require the intervention of a hair stylist.

The at least one reducing agent is then left to act for a time that is sufficient to obtain the desired reduction. This leave-in time may, for example, range from 2 to 30 minutes and such as from 5 to 15 minutes.

It is possible to insert an intermediate rinsing step between the reduction step (c) and the oxidation step (d). However, care should be taken to ensure that this rinsing leaves a sufficient amount of precipitate to impose a shape on the hair during the oxidation step.

In one aspect of the present disclosure, the process does not comprise an intermediate rinsing step (between the reduction step (c) and the oxidation step (d)). The reason for this is that such a rinsing operation might weaken or remove the deposit of precipitated polymer, thus destroying the shaping of the head of hair during the fixing step.

The fixing composition is thus, for example, applied to the hair directly after the first leave-in stage. This application may take place as far as is possible without changing the shape imposed on the head of hair in the preceding step. Such an application may take place, for example, using an aerosol device that dispenses a fixing mousse or fine droplets of a fixing solution. The nature of the at least one oxidizing agent used in this fixing composition may not be a deciding factor for the present disclosure, and any known oxidizing agent usually used for oxidizing the hair may be used, such as hydrogen peroxide, urea peroxide, bromates, persalts, and mixtures thereof.

The leave-in time for the fixing step may range from 2 to 30 minutes and, for example, from 2 to 15 minutes.

The composition remaining on the hair is then removed by rinsing, optionally followed by washing and/or another hair treatment.

Each of the three compositions of the multi-component agent may also comprise active principles and cosmetic adjuvants commonly used in haircare. These additives are chosen, for example, from vitamins; amino acids; oligopeptides; peptides; hydrolyzed and non-hydrolyzed, modified and unmodified proteins; enzymes; branched and unbranched fatty acids; branched and unbranched fatty alcohols, animal waxes, plant waxes, mineral waxes, ceramides, pseudoceramides, hydroxylated organic acids, UV-screening agents, antioxidants, free-radical scavengers, chelating agents, antidandruff agents, seborrhoea regulators, calmatives, cationic surfactants, anionic surfactants, nonionic surfactants, amphoteric surfactants, cationic polymers, anionic polymers, neutral polymers, amphoteric polymers, organomodified silicones, non-organomodified silicones, mineral oils, plant oils, animal oils, polyisobutenes, poly(α-olefins), fatty esters, and hair dyes such as, direct dyes and pigments.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples below serve to illustrate the present disclosure in a non-limiting manner. For instance, the example below illustrates a multi-component agent for reshaping the hair.

### EXAMPLE

First Component

| | | |
|---|---|---|
| Luviset Si-P.U.R.A | 66.6 | g |
| Demineralized water | qs 100 | g |

Second Component

| Thioglycolic acid | 5.1 | g |
|---|---|---|
| Pentasodium salt of diethylenetriaminepentaacetic acid | 0.4 | g |
| Aqueous ammonia | 5.6 | g |
| Ammonium chloride | 5.0 | g |
| Hydroxypropyl guar | 0.8 | g |
| Mexomer ® PO* (Chimex) | 1.6 | g |
| Tegobetaine ® HS** (Goldschmidt) | 1.4 | g |
| Ammonium dithiodiglycolate | 1.6 | g |
| Glycerol | 3.0 | g |
| Fragrance, peptizer | q.s. | |
| Demineralized water | qs 100 | g |

*polymer of N,N,N,N-tetramethylhexamethylenediamine and of trimethylene chloride (INCI name: hexadimethrine chloride)
**cocamidopropylbetaine and glyceryl laurate

Third Component (Fixing Composition)

| 50% hydrogen peroxide solution | 4.8 | g |
|---|---|---|
| Stabilizers | 0.03 | g |
| Ammonium lauryl sulfate | 4.0 | g |
| Citric acid | qs pH 3 | |
| Fragrance, peptizer | q.s. | |
| Demineralized water | qs 100 | g |

These three components were used in accordance with the process of the present disclosure for permanently reshaping the hair. On all the models, the treated hair had texture and mass. It was easy to style, modelable and had body.

The cosmetic effects observed remained for a long time over a large number of shampoo washes.

When the process of the present disclosure was used on hair that has been dyed beforehand, the coloration did not suffer from the permanent-reshaping treatment.

1. A process for permanently reshaping hair, comprising:
(a) applying, to an area to be reshaped, a solution comprising at least one polymer chosen from anionic and amphoteric polymers in a medium comprising water and optionally, at least one cosmetically acceptable solvent,
(b) applying, to the area treated in step (a), a reducing composition comprising at least one reducing agent that is capable of cleaving disulfide linkages of the hair and a sufficient amount of ions chosen from metal cations and ammonium ions to precipitate in situ the at least one polymer,
(c) shaping the hair in the area treated, followed by a leave-in time for the reducing composition,
(d) applying, to the area treated in steps (a) and (b), a fixing composition comprising at least one oxidizing agent, followed by a leave-in time for the fixing composition, and
(e) rinsing the hair.

2. The process according to claim 1, further comprising rinsing the hair between steps (c) and (d) and leaving a sufficient amount of precipitate on the hair to impose a shape on the hair during the step (d).

3. The process according to claim 1, wherein the anionic polymers comprise a synthetic copolymer obtained by polyaddition of a mixture of monomers chosen from anionic and nonionic monomers.

4. The process according to claim 1, wherein the amphoteric polymers comprise a synthetic copolymer obtained by polyaddition of a mixture of monomers chosen from anionic, non-ionic, and cationic monomers.

5. The process according to claim 3, wherein the anionic monomers are chosen from acrylic acids, methacrylic acids, 2-acrylamidomethylpropanesulfonic acids, crotonic acids, and itaconic acids.

6. The process according to claim 3, wherein the nonionic monomers are chosen from C₁₋₄ alkyl acrylates, C₁₋₄ alkyl methacrylates, acrylamides, vinyl acetates, vinyl para-tert-butylbenzoates, methacrylamides, N-mono(C₁₋₈ alkyl)acrylamides, N-vinylformamides, N-vinylacetonamides, hydroxypropyl acrylates, and hydroxypropyl methacrylates.

7. The process according to claim 4, wherein the anionic monomers are chosen from acrylic acids, methacrylic acids, 2-acrylamidomethylpropanesulfonic acids, crotonic acids, and itaconic acids.

8. The process according to claim 4, wherein the nonionic monomers are chosen from C₁₋₄ alkyl acrylates, C₁₋₄ alkyl methacrylates, acrylamides, vinyl acetates, vinyl para-tert-butylbenzoates, methacrylamides, N-mono(C₁₋₈ alkyl)acrylamides, N-vinylformamides, N-vinylacetonamides, hydroxypropyl acrylates, and hydroxypropyl methacrylates.

9. The process according to claim 4, wherein the cationic monomers are chosen from di(C₁₋₄ alkyl)diallylammonium salts and the compounds of formula (I) 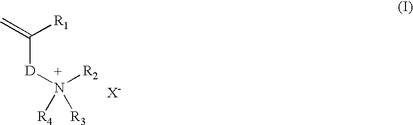
wherein
R₁ is chosen from a hydrogen atom and a methyl group,
R₂ and R₃, which may be identical or different, are chosen from hydrogen atoms and linear and branched C₁₋₄ alkyl groups,
R₄ is chosen from a hydrogen atom, a linear and branched C₁₋₄ alkyl group, and an aryl group,
D comprises the following unit:
YₙA
wherein
Y is chosen from amide, ester, urethane and urea functions,
A is chosen from linear, branched and cyclic C₁₋₁₀ alkylene groups, optionally substituted or interrupted with a ring chosen from divalent aromatic and heteroaromatic rings, optionally interrupted with a hetero atom chosen from O, N, S and P, optionally comprising at least one function chosen from ketone, amide, ester, urethane and urea functions,
n is 0 or 1, and
X⁻ is an anionic counterion.

10. The process according to claim 9, wherein X⁻ is chosen from a halide group and a sulphate anion.

11. The process according to claim 1, wherein the at least one polymer is a copolymer chosen from a copolymer of (meth)acrylic acid and of a C₁₋₆ alkyl (meth)acrylate, a vinyl acetate/vinyl para-tert-butylbenzoate/crotonic acid copolymer, and a copolymer of octylacrylamide, of tert-butylaminoethyl methacrylate and of at least one monomer chosen from acrylic and methacrylic acids and C₁₋₆ alkyl esters thereof.

12. The process according to claim 1, wherein the at least one polymer is a polycondensate.

13. The process according to claim 10, wherein the at least one polymer is a polyurethane.

14. The process according to claim 13, wherein said polyurethane is a silicone polyurethane.

15. The process according to claim 1, wherein the at least one polymer in step (a) is present in an amount at least equal to 5%, by weight relative to the total weight of the solution.

16. The process according to claim 15, wherein the at least one polymer in step (a) is present in an amount ranging from 7% to 30%, by weight relative to the total weight of the solution.

17. The process according to claim 1, wherein the ions are introduced by adding a salt chosen from mineral, organic and hydroxide salts, and optionally are provided by the at least one reducing agent when in salt form.

18. The process according to claim 1, wherein the ions in the reducing composition are present in an amount greater than 0.3 M.

19. The process according to claim 18, wherein the ions in the reducing composition are present in an amount ranging from 0.5 to 6 M.

20. The process according to claim 1, wherein the at least one reducing agent is chosen from sulfites, bisulfites, and thiols.

21. The process according to claim 20, wherein the at least one reducing agent is chosen from cysteines, cysteamines, thiolactic acids, thioglycolic acids, and cosmetically acceptable salts thereof.

22. The process according to claim 21, wherein the at least one reducing agent is ammonium thioglycolate.

23. The process according to claim 1, wherein the at least one reducing agent is present in an amount ranging from 1% to 25%, by weight relative to the total weight of the reducing composition.

24. The process according to claim 23, wherein the at least one reducing agent is present in an amount ranging from 1% to 10%, by weight relative to the total weight of the reducing composition.

25. The process according to claim 1, wherein the leave-in time for the reducing composition ranges from 2 to 30 minutes.

26. The process according to claim 25, wherein the leave-in time for the reducing composition ranges from 5 to 15 minutes.

27. The process according to claim 1, wherein the at least one oxidizing agent in the fixing composition is chosen from hydrogen peroxides, urea peroxides, bromates, persalts, and mixtures thereof.

28. The process according to claim 1, wherein the leave-in time for the fixing composition ranges from 2 to 30 minutes.

29. The process according to claim 28, wherein the leave-in time for the fixing composition ranges from 2 to 15 minutes.

30. A multi-component agent for permanently reshaping hair comprising
a first component comprising a solution comprising at least one polymer chosen from anionic and amphoteric polymers in a medium comprising water and, optionally, at least one cosmetically acceptable solvent that is capable of forming a solid precipitate in the presence of a sufficient amount of ions chosen from metal cations and ammonium ions,
a second component comprising a reducing composition comprising at least one reducing agent that is capable of cleaving disulfide linkages of the hair, and a sufficient amount of ions chosen from metal cations and ammonium ions to precipitate the at least one polymer of the first component, and
a third component comprising an oxidizing composition comprising at least one oxidizing agent that is capable of re-establishing disulfide linkages of the hair.

31. A multi-compartment kit for permanently reshaping hair comprising
a first compartment comprising a solution comprising at least one polymer chosen from anionic and amphoteric polymers in a medium comprising water and, optionally, at least one cosmetically acceptable solvent that is capable of forming a solid precipitate in the presence of a sufficient amount of ions chosen from metal cations and ammonium ions,
a second compartment comprising a reducing composition comprising at least one reducing agent that is capable of cleaving disulfide linkages of the hair, and a sufficient amount of ions chosen from metal cations and ammonium ions to precipitate the at least one polymer of the first component, and
a third compartment comprising an oxidizing composition comprising at least one oxidizing agent that is capable of re-establishing disulfide linkages of the hair.
